SEQUENCE LISTING (24 sequences)

SEQ ID NO: 1 - DNA, 497 bp, human
aatttttttttttttaagcggtttttattttttttttttgttttgtgaagcgggtgtgtaagtttcgggatcgtagcggttttagggaattttttttcgcgatgtttcggcgcgttagttcgttgcgtatatttcgttgcggttttttttttgttgtttgtttattttttaggtttcgttggggatttgggaaagagggaaaggttttttcggttagttgcgcggcgatttcggggattttagggcgtttttttgcggtcgacgttcggggtgtagcggtcgtcggggttggggtcggcgggagttcgcgggattttttagaagagcggtcggcgtcgtgatttagtattggggcggagcggggcgggattatttttataaggttcggaggtcgcgaggttttcgttggagtttcgtcgtcgtagttttcgttattagtgagtacgcgcggttcgtttttcggggatggggtttagagtttttagtatggggttaat

SEQ ID NO: 2 - DNA, 106 bp, human
cggggcgggattatttttataaggttcggaggtcgcgaggttttcgttggagtttcgtcgtcgtagttttcgttattagtgagtacgcgcggttcgcgttttcggg

SEQ ID NO: 3 - DNA, 106 bp, human; identical to SEQ ID NO: 2

SEQ ID NO: 4 - DNA, 106 bp, human; identical to SEQ ID NO: 2

SEQ ID NO: 5 - DNA, 106 bp, human; identical to SEQ ID NO: 2

SEQ ID NO: 6 - DNA, 106 bp, human
cggggcgggattatttttataaggttcggaggtcgcgaggttttcgttggagcttcgtcgtcgtagttttcgttattagtgagtacgcgcggttcgcgttttcggg

SEQ ID NO: 7 - DNA, 86 bp, human
tgtagtggttgttggggttggggttggtgggagtttgtgggattttttagaagagtggttggtgttgtgatttagtattggggcgg

SEQ ID NO: 8 - DNA, 86 bp, human
tgtagtggtcgtcggggttggggtcggtgggagttcgcgggattttttaggagagcggtcggcgtcgtgatttagtattggggcgg

SEQ ID NO: 9 - DNA, 86 bp, human
tgtagcggtcgtcggggttagggccggcgggagttcgcgggattttttagaagagcggtcggcgtcgtgatttagtattggggcgg

SEQ ID NO: 10 - DNA, 86 bp, human
tgtagcggtcgtcggggttggggtcggcgggagttcgcgggattttttagaagagcggtcggcgtcgtgatttagtattggggcgg

SEQ ID NO: 11 - DNA, 86 bp, human; identical to SEQ ID NO: 10

SEQ ID NO: 12 - DNA, 92 bp, human
tgaagtggttgtgtaagttttgggattgtagtggttttagggaatttttttttgtgatgttttggtgtgttagtagttgtgtatattttgtt

SEQ ID NO: 13 - DNA, 92 bp, human; identical to SEQ ID NO: 12

SEQ ID NO: 14 - DNA, 92 bp, human
  misc_feature (4)..(4): n is a, c, g, or t
tgangcggtngngnaagtttcgggatcgnngcggtttnngggaatttttttntgcgatgtttcggcgcgatagtagttgtgtatatttcgtt

SEQ ID NO: 15 - DNA, 92 bp, human
tgaagcggttgtgtaagtttcgggatcgtagcggttttagggaatttttttttgcgatgtttcggcgcgttagtagttgtgtatatttcgtt

SEQ ID NO: 16 - DNA, 92 bp, human
tgaagcggttgtgtaagtttcgggatcgtagcggttttagggaattttttttcgcgatgtttcggcgcgttagtagttgtgtatatttcgtt

SEQ ID NO: 17 - DNA, 47 bp, human
cgcacggcgaactcccgccgacgtgcgtgtagcggtcgtcggggttg

SEQ ID NO: 18 - DNA, 22 bp, human
gccccaatactaaatcacgacg

SEQ ID NO: 19 - DNA, 44 bp, human
ccggtcgcgaggttttcgaccggccgaaaaacgaaccgcgcgta

SEQ ID NO: 20 - DNA, 27 bp, human
gggcgggattatttttataaggttcgg

SEQ ID NO: 21 - DNA, 48 bp, human
cggccctaaaaccgctacgagggccggaagcgggtgtgtaagtttcgg

SEQ ID NO: 22 - DNA, 26 bp, human
acgaaatatacgcaacgaactaacgc

SEQ ID NO: 23 - DNA, 52 bp, human
ccgcgcatcaccaccccacacgcgcggggagtatataggttggggaagtttg

SEQ ID NO: 24 - DNA, 27 bp, human
aacacacaataacaaacacaaattcac

(19) United States
(12) Patent Application Publication
(10) Pub. No.: US 2008/0213781 A1
(43) Pub. Date: Sep. 4, 2008

(54) Methods of detecting methylation patterns within a CpG island

(76) Inventors: Jonathan F. Baden, Bridgewater, NJ (US); Jennifer Painter, Piscataway, NJ (US); Shobha A. Varde, Jacksonville, FL (US); Jyoti Mehrotra, Bridgewater, NJ (US); Tatiana I. Vener, Stirling, NJ (US); Abhijit Mazumder, Basking Ridge, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/032,085

(22) Filed: Feb. 15, 2008

Related U.S. Application Data
(60) Provisional application No. 60/889,944, filed on Feb. 15, 2007.

(51) Int. Cl. C12Q 1/68

(52) U.S. Cl. 435/6

(57) ABSTRACT

A method of increasing sensitivity of a DNA methylation assay by determining complementation within a CpG island of the methylated DNA.

### STATEMENT REGARDING FEDERALLY SPONSORED RESEARCH OR DEVELOPMENT

No government funds were used to make this invention.

### REFERENCE TO SEQUENCE LISTING, OR A COMPUTER PROGRAM LISTING COMPACT DISK APPENDIX

Reference to a “Sequence Listing”, a table, or a computer program listing appendix submitted on a compact disc and an incorporation by reference of the material on the compact disc including duplicates and the files on each compact disc.

### BACKGROUND OF THE INVENTION

Epigenetic changes (alterations in gene expression that do not involve alterations in DNA nucleotide sequences) are primarily comprised of modifications in DNA methylation and remodeling of chromatin. Alterations in DNA methylation have been documented in a wide range of tumors and genes. Esteller et al. (2001); Bastian et al. (2004); and Esteller (2005). The extent of methylation at a particular CpG site can vary across patient samples. Jeronimo et al. (2001); and Pao et al (2001).

A number of potential methylation markers have recently been disclosed. Glutathione S-transferases (GSTs) are exemplary proteins in which the methylation status of the genes that express them can have important prognostic and diagnostic value for prostate cancer. The proteins catalyze intracellular detoxification reactions, including the inactivation of electrophilic carcinogens, by conjugating chemically-reactive electrophiles to glutathione. (Pickett et al. (1989); Coles et al. (1990); and Rushmore et al. (1993). Human GSTs, encoded by several different genes at different loci, have been classified into four families referred to as alpha, mu, pi, and theta. Mannervik et al. (1992). Decreased GSTP1 expression resulting from epigenetic changes is often related to prostate and hepatic cancers.

In addition, computational approaches (Das et al. (2006)) and bisulfite sequencing (Chan et al. (2005)) indicate that multiple sites within a CpG island can be methylated and that the extent of methylation can vary across these sites. For example, in oral cancer, differences in the degree of methylation of individual CpG sites were noted for p16, E-cadherin, cyclin A1, and cytoglobin. Shaw et al. (2006). In prostate and bladder tumors, the endothelin receptor B displayed hotspots for methylation. (Pao et al. (2001). In colorectal and gastric cancer, methylation of the edge of the CpG island of the death-associated protein kinase gene was detected in virtually every sample, in contrast to the more central regions. Satoh et al. (2002). The differential distribution of methylation is found the RASSF1A CpG island in breast cancer and methylation may progressively spread from the first exon into the promoter area. Yan et al. (2003); and Strunnikova et al. (2005). RASSF2 has frequent methylation at the 5′ and 3′ edges of the CpG island, with less frequent methylation near the transcription start site. Endoh et al. (2005).

In endometrial carcinoma four GSTP1 designs showed sensitivities between 14% and 24% but the sample sizes were too small to determine if these differences were real. (Chan et al. 2005). Two assay designs increase sensitivity of detection of prostate carcinoma (Nakayama et al. (2003)); however, both designs shared the same reverse primer so there was considerable overlap in the regions interrogated. Differences exist in the percent methylation for different CpG sequences for p16, E-cadherin, cyclin A1, and cytoglobin. Shaw et al. (2006). Differential methylation levels at CpG sites exist in breast cancer. Yan et al. (2003).

An inverse correlation exists between tumor MLH1 RNA expression and MLH1 DNA methylation. Yu et al. (2006). Methylation-positive samples exhibited lower levels of RNA expression of the DAPK gene in lung cancer cell lines. Toyooka et al. (2003). However, those studies examined only one site of methylation so correlations with RNA expression at multiple locations in a CpG island could not be determined. The core region surrounding the transcription start site is an informative surrogate for promoter methylation. Eckhardt et al. (2006).

In squamous cell carcinoma of the esophagus, methylation at individual genes increased in frequency from normal to invasive cancer. (Guo et al. 2006). Methylation of TMS1 (p=0.002), DcR1 (p=−0.01), DcR2 (p=0.03), and CRBP1 (p=0.03) correlate with Gleason score and methylation of CRBP1 correlates with higher stage (p=0.0002) and methylation of Reprimo (p=0.02) and TMS1 (p=0.006) correlated with higher (>8 ng/ml) PSA levels. Suzuki et al. (2006). Methylation status was correlated with the extent of myometrial invasion in endometrial carcinoma. A significantly (p=0.04) higher frequency of ASC methylation in the tumor-adjacent, normal tissue for patients was associated with biochemical recurrence, suggesting a correlation with aggressive disease. Chan et al. (2005). RARb2, PTGS2, and EDNRB may have prognostic value in patients undergoing radical prostatectomy. Bastian et al. (2006).

Methylation-specific PCR (MSP) assays have been performed at multiple sites of two genes known to be methylated in prostate cancer, GSTP1 and RARb2. Lee et al. (1994); Harden et al. (2003); Jeronimo et al. (2004); and Nakayama et al. (2001).

### BRIEF DESCRIPTION OF THE DRAWINGS

FIG. 1 shows sequences of the MSP Scorpion designs. The designs are shown relative to their location in the CpG island. The sequences of the forward primer, Scorpion, and reverse primer for each design are shaded in the sequence.

FIG. 2 shows Bisulfite sequencing data from representative clones for the GSTP1 (−46) region (A), GSTP1 (−158) region (B), and GSTP1 (−390) region (C). A sample (x) and its clone number (y) is denoted as x-y. Methylated CG nucleotides are shown in red while unmethylated CG nucleotides are shown as TG sequences (after bisulfite and PCR) and are in blue. Boxes depict the locations of the forward primer, probe, and reverse primer.

FIG. 3 shows the titration curves depicting the analytical sensitivity of the GSTP1 (−46) design (A), GSTP1 (−158) design (B), and the GSTP1 (−390) design (C). Methylated DNA was spiked into unmethylated DNA in a serial dilution experiment and MSP was performed on bisulfite-modified DNA. Each data point is the average of 5 replicates. The error bars depict one standard deviation.

FIG. 4 shows the clinical sensitivity and specificity of the three GSTP1 designs. (A) Scatterplot of adenocarcinoma and benign samples showing the copies of each methylated GSTP1 design. (B) Heat map depicting the sensitivity and specificity of the three different GSTP1 designs. Red depicts higher methylation levels and green depicts the absence of methylation.

FIG. 5 shows the correlation of the extent of methylation and the expression of the GSTP1 transcript in 9 cancer samples for the three different GSTP1 designs. (A) Scatterplot depicting relative transcript level for the GSTP1 gene versus the methylation status for each sample. The filled circle denotes the presence of methylation in the design region and the open circle denotes the absence of methylation in the design region. (B) Scatterplot depicting methylation ratio of the GSTP1 (−46) region versus the relative expression level of the GSTP1 gene.

### DESCRIPTION OF THE INVENTION

It has now been shown that epigenetic complementation can be achieved within a CpG island of a gene and that interrogation of different sites could provide a more complete molecular portrait of a tumor.

DNA methylation of CpG dinucleotides can occur in a heterogeneous pattern. Several studies have shown that methylation is more prevalent at the edges of CpG islands. We designed and evaluated three different methylation-specific PCR (MSP) assays for GSTP1. The MSP data showed a strong correlation with bisulfite sequencing of these regions. Three designs demonstrated different clinical sensitivities (for detection of adenocarcinomas) and different levels of methylation; moreover, simultaneous use of two designs enabled higher sensitivity because the two designs detected overlapping samples. Importantly, all three GSTP1 assay designs demonstrated higher assay sensitivity for prostate cancers having a higher Gleason score but the reduction in sensitivity for detection of prostate cancers having a lower Gleason score was significant for one of the designs. Lastly, the three designs showed different correlations with RNA expression levels. Therefore, the use of epigenetic complementation within a CpG island of a gene, to increase sensitivity, can be coupled with complementation derived from the use of multiple markers to further increase the performance of new diagnostic assays in oncology.

A Biomarker is any indicia of an indicated Marker nucleic acid/protein. Nucleic acids can be any known in the art including, without limitation, nuclear, mitochondrial (homeoplasmy, heteroplasmy), viral, bacterial, fungal, mycoplasmal, etc. The indicia can be direct or indirect and measure over- or under-expression of the gene given the physiologic parameters and in comparison to an internal control, placebo, normal tissue or another carcinoma. Biomarkers include, without limitation, nucleic acids and proteins (both over and under-expression and direct and indirect). Using nucleic acids as Biomarkers can include any method known in the art including, without limitation, measuring DNA amplification, deletion, insertion, duplication, RNA, microRNA (miRNA), loss of heterozygosity (LOH), single nucleotide polymorphisms (SNPs, Brookes (1999)), copy number polymorphisms (CNPs) either directly or upon genome amplification, microsatellite DNA, epigenetic changes such as DNA hypo- or hyper-methylation and FISH. Using proteins as Biomarkers includes any method known in the art including, without limitation, measuring amount, activity, modifications such as glycosylation, phosphorylation, ADP-ribosylation, ubiquitination, etc., or imunohistochemistry (IHC) and turnover. Other Biomarkers include imaging, molecular profiling, cell count and apoptosis Markers.

A Marker gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

The inventive methods, compositions, articles, and kits of described and claimed in this specification include one or more Marker genes. “Marker” or “Marker gene” is used throughout this specification to refer to genes and gene expression products that correspond with any gene the over- or under-expression of which is associated with an indication or tissue type.

The inventive methods, compositions, articles, and kits of described and claimed in this specification include one or more Marker genes. “Marker” or “Marker gene” is used throughout this specification to refer to genes and gene expression products that correspond with any gene the over- or under-expression of which is associated with an indication or tissue type.

The modification of nucleic acid sequences having the potential to express proteins, peptides, or mRNA (such sequences referred to as “genes”) within the genome has been shown, by itself, to be determinative of whether a protein, peptide, or mRNA is expressed in a given cell. Whether or not a given gene capable of expressing proteins, peptides, or mRNA does so and to what extent such expression occurs, if at all, is determined by a variety of complex factors. Irrespective of difficulties in understanding and assessing these factors, assaying gene expression or modification patterns can provide useful information about the occurrence of important events such as tumorogenesis, metastasis, apoptosis, and other clinically relevant phenomena. Relative indications of the degree to which genes are active or inactive can be found in gene expression or modification profiles.

A sample can be any biological fluid, cell, tissue, organ or portion thereof that contains genomic DNA suitable for methylation detection. A test sample can include or be suspected to include a neoplastic cell, such as a cell from the colon, rectum, breast, ovary, prostate, kidney, lung, blood, brain or other organ or tissue that contains or is suspected to contain a neoplastic cell. The term includes samples present in an individual as well as samples obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid molecule or protein preparation. A reference sample can be used to establish a reference level and, accordingly, can be derived from the source tissue that meets having the particular phenotypic characteristics to which the test sample is to be compared.

A sample for determining gene modification profiles can be obtained by any method known in the art. Samples can be obtained according to standard techniques from all types of biological sources that are usual sources of genomic DNA including, but not limited to cells or cellular components which contain DNA, cell lines, biopsies, bodily fluids such as blood, sputum, stool, urine, cerebrospinal fluid, ejaculate, tissue embedded in paraffin such as tissue from eyes, intestine, kidney, brain, heart, prostate, lung, breast or liver, histological object slides, and all possible combinations thereof. A suitable biological sample can be sourced and acquired subsequent to the formulation of the diagnostic aim of the marker. A sample can be derived from a population of cells or from a tissue that is predicted to be afflicted with or phenotypic of the condition. The genomic DNA can be derived from a high-quality source such that the sample contains only the tissue type of interest, minimum contamination and minimum DNA fragmentation.

Sample preparation requires the collection of patient samples. Patient samples used in the inventive method are those that are suspected of containing diseased cells such as epithelial cells taken from the primary tumor in a colon sample or from surgical margins. Laser Capture Microdissection (LCM) technology is one way to select the cells to be studied, minimizing variability caused by cell type heterogeneity. Consequently, moderate or small changes in gene expression between normal and cancerous cells can be readily detected. Samples can also comprise circulating epithelial cells extracted from peripheral blood. These can be obtained according to a number of methods but the most preferred method is the magnetic separation technique described in U.S. Pat. No. 6,136,182. Once the sample containing the cells of interest has been obtained, DNA is extracted and amplified and a cytosine methylation profile is obtained, for genes in the appropriate portfolios.

DNA methylation and methods related thereto are discussed for instance in U.S. patent publication numbers 20020197639, 20030022215, 20030032026, 20030082600, 20030087258, 20030096289, 20030129620, 20030148290, 20030157510, 20030170684, 20030215842, 20030224040, 20030232351, 20040023279, 20040038245, 20040048275, 20040072197, 20040086944, 20040101843, 20040115663, 20040132048, 20040137474, 20040146866, 20040146868, 20040152080, 20040171118, 20040203048, 20040241704, 20040248090, 20040248120, 20040265814, 20050009059, 20050019762, 20050026183, 20050053937, 20050064428, 20050069879, 20050079527, 20050089870, 20050130172, 20050153296, 20050196792, 20050208491, 20050208538, 20050214812, 20050233340, 20050239101, 20050260630, 20050266458, 20050287553 and U.S. Pat. Nos. 5,786,146, 6,214,556, 6,251,594, 6,331,393 and 6,335,165.

DNA modification kits are commercially available, they convert purified genomic DNA with unmethylated cytosines into genomic lacking unmethylated cytosines but with additional uracils. The treatment is a two-step chemical process consisting a deamination reaction facilitated by bisulfite and a desulfonation step facilitated by sodium hydroxide. Typically the deamination reaction is performed as a liquid and is terminated by incubation on ice followed by adding column binding buffer. Following solid phase binding and washing the DNA is eluted and the desulfonation reaction is performed in a liquid. Adding ethanol terminates the reaction and the modified DNA is cleaned up by precipitation. However, both commercially available kits (Zymo and Chemicon) perform the desulfonation reaction while the DNA is bound on the column and washing the column terminates the reaction. The treated DNA is eluted from the column ready for MSP assay.

The step of isolating DNA may be conducted in accordance with standard protocols. The DNA may be isolated from any suitable body sample, such as cells from tissue (fresh or fixed samples), blood (including serum and plasma), semen, urine, lymph or bone marrow. For some types of body samples, particularly fluid samples such as blood, semen, urine and lymph, it may be preferred to firstly subject the sample to a process to enrich the concentration of a certain cell type (e.g. prostate cells). One suitable process for enrichment involves the separation of required cells through the use of cell-specific antibodies coupled to magnetic beads and a magnetic cell separation device.

Prior to the amplifying step, the isolated DNA is preferably treated such that unmethylated cytosines are converted to uracil or another nucleotide capable of forming a base pair with adenine while methylated cytosines are unchanged or are converted to a nucleotide capable of forming a base pair with guanine.

Preferably, following treatment and amplification of the isolated DNA, a test is performed to verify that unmethylated cytosines have been efficiently converted to uracil or another nucleotide capable of forming a base pair with adenine, and that methylated cytosines have remained unchanged or efficiently converted to another nucleotide capable of forming a base pair with guanine.

Preferably, the treatment of the isolated DNA involves reacting the isolated DNA with bisulphite in accordance with standard protocols. In bisulphite treatment, unmethylated cytosines are converted to uracil whereas methylated cytosines will be unchanged. Verification that unmethylated cytosines have been converted to uracil and that methylated cystosines have remained unchanged may be achieved by; (i) restricting an aliquot of the treated and amplified DNA with a suitable restriction enzyme which recognize a restriction site generated by or resistant to the bisulphite treatment, and (ii) assessing the restriction fragment pattern by electrophoresis. Alternatively, verification may be achieved by differential hybridization using specific oligonucleotides targeted to regions of the treated DNA where unmethylated cytosines would have been converted to uracil and methylated cytosines would have remained unchanged.

The amplifying step may involve polymerase chain reaction (PCR) amplification, ligase chain reaction amplification and others. Stirzaker et al. (1997); and Tremblay et al. (1997).

Preferably, the amplifying step is conducted in accordance with standard protocols for PCR amplification, in which case, the reactants will typically be suitable primers, dNTPs and a thermostable DNA polymerase, and the conditions will be cycles of varying temperatures and durations to effect alternating denaturation of strand duplexes, annealing of primers (e.g. under high stringency conditions) and subsequent DNA synthesis.

To achieve selective PCR amplification with bisulphite-treated DNA, primers and conditions may be used to discriminate between a target region including a site or sites of abnormal cytosine methylation and a target region where there is no site or sites of abnormal cytosine methylation. Thus, for amplification only of a target region where the said site or sites at which abnormal cytosine methylation occurs is/are methylated, the primers used to anneal to the bisulphite-treated DNA (i.e. reverse primers) may include a guanine nucleotide at a site at which it will form a base pair with a methylated cytosine. Such primers will form a mismatch if the target region in the isolated DNA has unmethylated cytosine nucleotide (which would have been converted to uracil by the bisulphite treatment) at the site or sites at which abnormal cytosine methylation occurs. The primers used for annealing to the opposite strand (i.e. the forward primers) may include a cytosine nucleotide at any site corresponding to site of methylated cytosine in the bisulphite-treated DNA.

The step of amplifying is used to amplify a target region within the GST-Pi gene and/or its regulatory flanking sequences. The regulatory flanking sequences may be regarded as the flanking sequences 5′ and 3′ of the GST-Pi gene which include the elements that regulate, either alone or in combination with another like element, expression of the GST-Pi gene.

Sites of abnormal cytosine methylation can be detected for the purposes of diagnosing or prognosing a disease or condition by methods which do not involve selective amplification. For instance, oligonucleotide/polynucleotide probes could be designed for use in hybridization studies (e.g. Southern blotting) with bisulphite-treated DNA which, under appropriate conditions of stringency, selectively hybridize only to DNA which includes a site or sites of abnormal methylation of cytosine. Alternatively, an appropriately selected informative restriction enzyme can be used to produce restriction fragment patterns that distinguish between DNA which does and does not include a site or sites of abnormal methylation of cytosine.

The method of the invention can also include contacting a nucleic acid-containing specimen with an agent that modifies unmethylated cytosine; amplifying the CpG containing nucleic acid in the specimen by means of CpG-specific oligonucleotide primers; and detecting the methylated nucleic acid. The preferred modification is the 15 conversion of unmethylated cytosines to another nucleotide that will distinguish the unmethylated from the methylated cytosine. Preferably, the agent modifies unmethylated cytosine to uracil and is sodium bisulfite, however, other agents that modify unmethylated cytosine, but not methylated cytosine can also be used. Sodium bisulfite (NaHSO₃) modification is most preferred and reacts readily with the 5,6-double bond of cytosine, but poorly with methylated cytosine. Cytosine reacts with the bisulfite ion to form a sulfonated cytosine reaction intermediate susceptible to deamination, giving rise to a sulfonated uracil. The sulfonate group can be removed under alkaline conditions, resulting in the formation of uracil. Uracil is recognized as a thymine by Taq polymerase and therefore upon PCR, the resultant product contains cytosine only at the position where 5-methylcytosine occurs in the starting template. Scorpion reporters and reagents and other detection systems similarly distinguish modified from unmodified species treated in this manner.

The primers used in the invention for amplification of a CpG-containing nucleic acid in the specimen, after modification (e.g., with bisulfite), specifically distinguish between untreated DNA, methylated, and non-methylated DNA. In methylation specific PCR (MSPCR), primers or priming sequences for the non-methylated DNA preferably have a T in the 3′ CG pair to distinguish it from the C retained in methylated DNA, and the complement is designed for the antisense primer. MSP primers or priming sequences for non-methylated DNA usually contain relatively few Cs or Gs in the sequence since the Cs will be absent in the sense primer and the Gs absent in the antisense primer (C becomes modified to U (uracil) which is amplified as T (thymidine) in the amplification product).

The primers of the invention are oligonucleotides of sufficient length and appropriate sequence so as to provide specific initiation of polymerization on a significant number of nucleic acids in the polymorphic locus. When exposed to appropriate probes or reporters, the sequences that are amplified reveal methylation status and thus diagnostic information. Preferred primers are most preferably eight or more deoxyribonucleotides or ribonucleotides capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The priming segment of the primer or priming sequence is preferably single stranded for maximum efficiency in amplification, but may be double stranded. If double stranded, the primer is first treated to separate its strands before being used to prepare extension products. The primer must be sufficiently long to prime the synthesis of extension products in the presence of the inducing agent for polymerization. The exact length of primer will depend on factors such as temperature, buffer, cations, and nucleotide composition. The oligonucleotide primers most preferably contain about 12-20 nucleotides although they may contain more or fewer nucleotides, preferably according to well known design guidelines or rules. Primers are designed to be substantially complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with the 5′ and 3′ flanking sequence(s) to hybridize and permit amplification of the genomic locus. The primers are employed in the amplification process. That is, reactions (preferably, an enzymatic chain reaction) that produce greater quantities of target locus relative to the number of reaction steps involved. In a most preferred embodiment, the reaction produces exponentially greater quantities of the target locus. Reactions such as these include the PCR reaction. Typically, one primer is complementary to the negative (−) strand of the locus and the other is complementary to the positive (+) strand. Annealing the primers to denatured nucleic acid followed by extension with an enzyme, such as the large fragment of DNA Polymerase I (Klenow) and nucleotides, results in newly synthesized + and − strands containing the target locus sequence. The product of the chain reaction is a discrete nucleic acid duplex with termini corresponding to the ends of the specific primers employed.

The primers may be prepared using any suitable method, such as conventional phosphotriester and phosphodiester methods including automated methods. In one such automated embodiment, diethylphosphoramidites are used as starting materials and may be synthesized as described by Beaucage et al. (1981). A method for synthesizing oligonucleotides on a modified solid support is described in U.S. Pat. No. 4,458,066.

Any nucleic acid specimen taken from urine or urethral wash, in purified or non-purified form, can be utilized as the starting nucleic acid or acids, provided it contains, or is suspected of containing, the specific nucleic acid sequence containing the target locus (e.g., CpG). Thus, the process may employ, for example, DNA or RNA, including messenger RNA. The DNA or RNA may be single stranded or double stranded. In the event that RNA is to be used as a template, enzymes, and/or conditions optimal for reverse transcribing the template to DNA would be utilized. In addition, a DNA-RNA hybrid containing one strand of each may be utilized. A mixture of nucleic acids may also be employed, or the nucleic acids produced in a previous amplification reaction herein, using the same or different primers may be so utilized. The specific nucleic acid sequence to be amplified, i.e., the target locus, may be a fraction of a larger molecule or can be present initially as a discrete molecule so that the specific sequence constitutes the entire nucleic acid.

If the extracted sample is impure, it may be treated before amplification with an amount of a reagent effective to open the cells, fluids, tissues, or animal cell membranes of the sample, and to expose and/or separate the strand(s) of the nucleic acid(s). This lysing and nucleic acid denaturing step to expose and separate the strands will allow amplification to occur much more readily.

Where the target nucleic acid sequence of the sample contains two strands, it is necessary to separate the strands of the nucleic acid before it can be used as the template. Strand separation can be effected either as a separate step or simultaneously with the synthesis of the primer extension products. This strand separation can be accomplished using various suitable denaturing conditions, including physical, chemical or enzymatic means. One physical method of separating nucleic acid strands involves heating the nucleic acid until it is denatured. Typical heat denaturation may involve temperatures ranging from about 80 to 105° C. for up to 10 minutes. Strand separation may also be induced by an enzyme from the class of enzymes known as helicases or by the enzyme RecA, which has helicase activity, and in the presence of riboATP, is known to denature DNA. Reaction conditions that are suitable for strand separation of nucleic acids using helicases are described by Kuhn Hoffmann-Berling (1978). Techniques for using RecA are reviewed in Radding (1982). Refinements of these techniques are now also well known.

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. A molar excess (for genomic nucleic acid, usually about 10₈:1, primer:template) of the two oligonucleotide primers is preferably added to the buffer containing the separated template strands. The amount of complementary strand may not be known if the process of the invention is used for diagnostic applications, so the amount of primer relative to the amount of complementary strand cannot always be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90-100° C. for up to 10 minutes, preferably from 1 to 4 minutes. After this heating period, the solution is allowed to cool to room temperature, which is preferable for the primer hybridization. To the cooled mixture is added an appropriate agent for effecting the primer extension reaction (the “agent for polymerization”), and the reaction is allowed to occur under conditions known in the art. The agent for polymerization may also be added together with the other reagents if it is heat stable. This synthesis (or amplification) reaction may occur at room temperature up to a temperature at which the agent for polymerization no longer functions. The agent for polymerization may be any compound or system that will function to accomplish the synthesis of primer extension products, preferably enzymes. Suitable enzymes for this purpose include, for example, E. coli DNA polymerase 1, Klenow fragment of E. coli DNA polymerase I, T4 DNA polymerase, other available DNA polymerases, polymerase mutants, reverse transcriptase, and other enzymes, including heat-stable enzymes (e.g., those enzymes which perform primer extension after being subjected to temperatures sufficiently elevated to cause denaturation). A preferred agent is Taq polymerase. Suitable enzymes will facilitate combination of the nucleotides in the proper manner to form the primer extension products complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

Most preferably, the method of amplifying is by PCR. Alternative methods of amplification can also be employed as long as the methylated and non-methylated loci amplified by PCR using the primers of the invention is similarly amplified by the alternative means. In one such most preferred embodiment, the assay is conducted as a nested PCR. In nested PCR methods, two or more staged polymerase chain reactions are undertaken. In a first-stage polymerase chain reaction, a pair of outer oligonucleotide primers, consisting of an upper and a lower primer that flank a particular first target nucleotide sequence in the 5′ and 3′ position, respectively, are used to amplify that first sequence. In subsequent stages, a second set of inner or nested oligonucleotide primers, also consisting of an upper and a lower primer, are used to amplify a smaller second target nucleotide sequence that is contained within the first target nucleotide sequence.

The upper and lower inner primers flank the second target nucleotide sequence in the 5′ and 3′ positions, respectively. Flanking primers are complementary to segments on the 3′-end portions of the double-stranded target nucleotide sequence that is amplified during the PCR process. The first nucleotide sequence within the region of the gene targeted for amplification in the first-stage polymerase chain reaction is flanked by an upper primer in the 5′ upstream position and a lower primer in the 3′ downstream position. The first targeted nucleotide sequence, and hence the amplification product of the first-stage polymerase chain reaction, has a predicted base-pair length, which is determined by the base-pair distance between the 5′ upstream and 3′ downstream hybridization positions of the upper and lower primers, respectively, of the outer primer pair.

At the end of the first-stage polymerase chain reaction, an aliquot of the resulting mixture is carried over into a second-stage polymerase chain reaction. This is preferably conducted within a sealed or closed vessel automatically such as with the “SMART CAP” device from Cepheid. In this second-stage reaction, the products of the first-stage reaction are combined with specific inner or nested primers. These inner primers are derived from nucleotide sequences within the first targeted nucleotide sequence and flank a second, smaller targeted nucleotide sequence contained within the first targeted nucleotide sequence. This mixture is subjected to initial denaturation, annealing, and extension steps, followed by thermocycling as before to allow for repeated denaturation, annealing, and extension or replication of the second targeted nucleotide sequence. This second targeted nucleotide sequence is flanked by an upper primer in the 5′ upstream position and a lower primer in the 3′ downstream position. The second targeted nucleotide sequence, and hence the amplification product of the second-stage PCR, also has a predicted base-pair length, which is determined by the base-pair distance between the 5′ upstream and 3′ downstream hybridization positions of the upper and lower primers, respectively, of the inner primer pair.

The amplified products are preferably identified as methylated or non-methylated with a probe or reporter specific to the product as described in U.S. Pat. No. 4,683,195. Advances in the field of probes and reporters for detecting polynucleotides are well known to those skilled in the art.

The kits of the invention can be configured with a variety of components provided that they all contain at least one primer or probe or a detection molecule (e.g., Scorpion reporter). In one embodiment, the kit includes reagents for amplifying and detecting hypermethylated Marker segments. Optionally, the kit includes sample preparation reagents and /or articles (e.g., tubes) to extract nucleic acids from samples.

In a preferred kit, necessary reagents are included such as, a corresponding PCR primer set, a thermostable DNA polymerase, such as Taq polymerase, and a suitable detection reagent(s) such as hydrolysis probe or molecular beacon. In optionally preferred kits, detection reagents are Scorpion reporters or reagents. A single dye primer or a fluorescent dye specific to double-stranded DNA such as ethidium bromide can also be used. The primers are preferably in quantities that yield high concentrations. Additional materials in the kit may include: suitable reaction tubes or vials, a barrier composition, typically a wax bead, optionally including magnesium; necessary buffers and reagents such as dNTPs; control nucleic acid(s) and/or any additional buffers, compounds, co-factors, ionic constituents, proteins and enzymes, polymers, and the like that may be used in MSP reactions. Optionally, the kits include nucleic acid extraction reagents and materials.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

The assays of the invention detect hypermethylation of nucleic acids that correspond to particular genes whose methylation status correlates with cancer. A nucleic acid corresponds to a gene whose methylation status correlates with cancer when methylation status of such a gene provides information about prostate cancer and the sequence is a coding portion of the gene or its complement, a representative portion of the gene or its complement, a promoter or regulatory sequence for the gene or its complement, a sequence that indicates the presence of the gene or its complement, or the full length sequence of the gene or its complement. Such nucleic acids are referred to as Markers in this specification. Markers correspond, without limitation, to the following genes GSTP1, APC, RARβ2, HINC1. Other sequences of interest include constitutive genes useful as assay controls such as beta-Actin and PTGS2.

Assays for detecting hypermethylation include such techniques as MSP and restriction endonuclease analysis. The promoter region is a particularly noteworthy target for detecting such hypermethylation analysis. Sequence analysis of the promoter region of GSTP1 shows that nearly 72% of the nucleotides are CG and about 10% are CpG dinucleotides.

The invention includes determining the methylation status of certain regions of the Markers in which the DNA associated with cancer is amplified and detected. Since a decreased level of the protein encoded by the Marker (i.e., less transcription) is often the result of hypermethylation of a particular region such as the promoter, it is desirable to determine whether such regions are hypermethylated. This is seen most demonstrably in the case of the GSTP1 gene. A nucleic acid probe or reporter specific for certain Marker regions is used to detect the presence of methylated regions of the Marker gene. Hypermethylated regions are those that are methylated to a statistically significant greater degree in samples from diseased tissue as compared to normal tissue.

The GSTP1 promoter is the most preferred Marker. It is a polynucleotide sequence that can direct transcription of the gene to produce a glutathione-s-transferase protein. The promoter region is located upstream, or 5′ to the structural gene. It may include elements which are sufficient to render promoter-dependent gene expression controllable for cell type specific, tissue-specific, or inducible by external signals or agents; such elements may be located in the 5′ or 3′ regions of the of the polynucleotide sequence.

One method of the invention includes contacting a target cell containing a Marker with a reagent that binds to the nucleic acid. The target cell component is a nucleic acid such as DNA extracted from urine by cell lysis and purification (column or solution based) yielding pure DNA that is devoid of proteins. The reagents include components that prime and probe PCR or MSP reactions and detect the target sequence. These reagents can include priming sequences combined with or bonded to their own reporter segments such as those referred to as Scorpion reagents or Scorpion reporters and described in U.S. Pat. Nos. 6,326,145 and 6,270,967. Though they are not the same, the terms “primers” and “priming sequences” may be used in this specification to refer to molecules or portions of molecules that prime the amplification of nucleic acid sequences.

One sensitive method of detecting methylation patterns involves combining the use of methylation-sensitive enzymes and the polymerase chain reaction (PCR). After digestion of DNA with the enzyme, PCR will amplify from primers flanking the restriction site only if DNA cleavage was prevented by methylation. The PCR primers of the invention are designed to target the promoter and transcription region that lies approximately between −71 and +59 bp according to genomic positioning number of M24485 (Genbank) from the transcription start site of GSTP1.

The following example is provided to illustrate but not limit the invention. All references cited herein are hereby incorporated herein by reference.

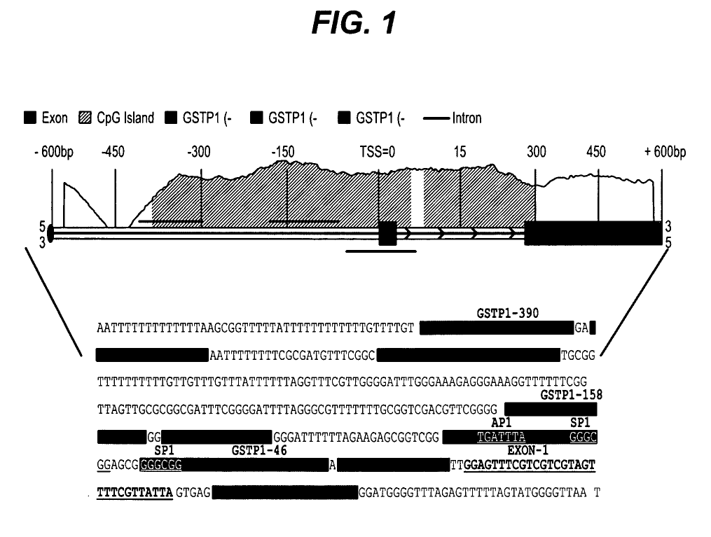

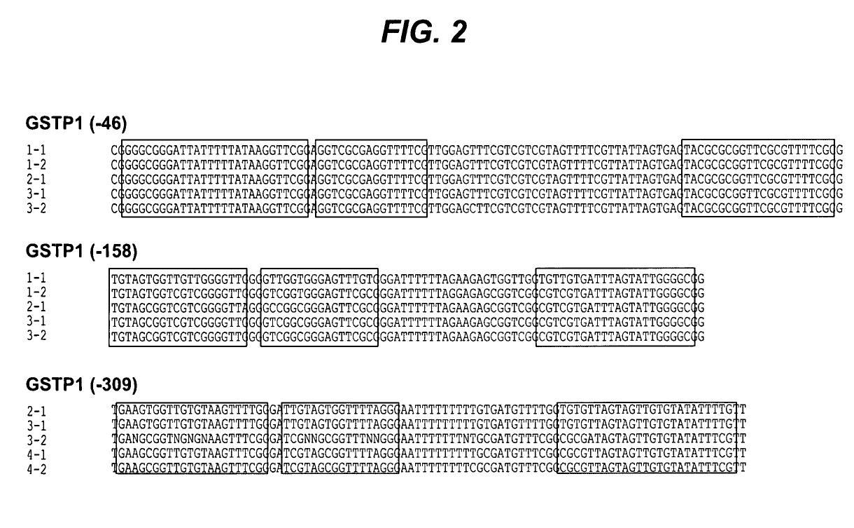

We designed three MSP assays for the GSTP1 gene. The sequences of a portion of the GSTP1 gene and the three MSP assays are shown in FIG. 1. Design GSTP1 (−390) is at the edge of the CpG island, design GSTP1 (−46) is at the transcription start site and design GSTP1 (−158) is centrally located within the CpG island. We analyzed methylation in these sequence regions using bisulfite sequencing (Table 1 and FIG. 2). In general, the data showed a good correlation between bisulfite sequencing and MSP. For example, sample 1 showed methylation in the GSTP1 (−46) region by PCR. Consistent with those findings, clonal sequencing demonstrated that a number of clones exhibited methylation in the GSTP1 (−46) region. However, sequencing also showed two clones which exhibited methylation in the GSTP1 (−158) region, which was not detected by PCR. Samples 2 and 3 showed methylation in both the GSTP1 (−46) and GSTP1 (−158) regions but not GSTP1 (−390) by PCR. Consistent with those findings, clonal sequencing demonstrated that a number of clones exhibited methylation in the GSTP1 (−46) and GSTP1 (−158) regions but, for the most part, not in the GSTP1 (−390) region. Sample 4 showed methylation in both the GSTP1 (−46) and GSTP1 (−390) regions but not GSTP1 (−158) by PCR. Consistent with those findings, clonal sequencing demonstrated that a number of clones exhibited methylation in the GSTP1 (−390) region. However, sequencing analysis failed to show methylation in the GSTP1 (−46) region, even though it was reported by PCR. We conclude that analysis of promoter and gene regions by either PCR or bisulfite sequencing can reveal differences in the extent of methylation across different sequences but that the different technologies may not always agree.

TABLE 1

| | | | |
|---|---|---|---|
| Sample | GSTP1 (−46) | GSTP1 (−158) | GSTP1 (−390) |

| | | | | | | |
|---|---|---|---|---|---|---|
| 1 | 1339 | ++ | — | + | — | — |
| 2 | 422 | ++ | 65 | ++ | — | — |
| 3 | 1251 | ++ | 2606 | ++ | — | + |
| 4 | 263 | — | — | — | 11445 | ++ |

Table 1 shows bisulfite sequencing data of cancer samples. For each sample, 20 clones were picked and sequenced for each of the designs. *Methylation ratio ([copies of GSTP1/copies of β-actin]×1000) as determined by Methylation-specific PCR;—denotes absence of a methylation signal. **++ denotes multiple clones exhibiting methylation; + denotes GSTP1 (−158) clones exhibiting methylation;—denotes absence of methylation in clones analyzed.

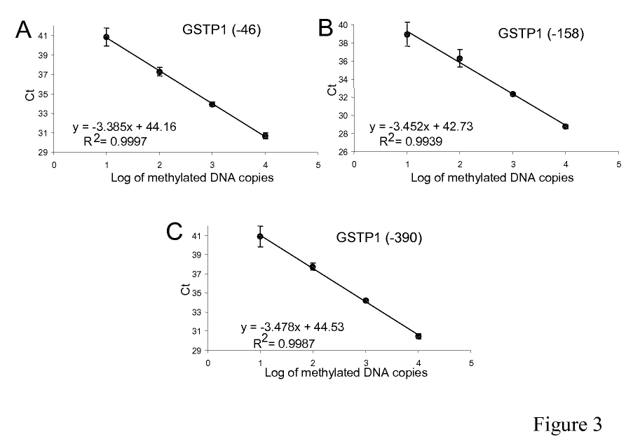

To ensure that these three GSTP1 designs had similar analytical sensitivities, we generated titration curves of methylated DNA spiked into unmethylated DNA for each of the three designs (FIG. 3). Each of the three designs showed excellent linearity from 10 to 10,000 copies of methylated DNA (R² value ranging from 0.9939 to 0.9997), high amplification efficiency (ranging from 96 to 98%), and good sensitivity. Furthermore, each assay showed excellent reproducibility in the PCR step, with a median coefficient of variation of 1% (ranging from 0.5 to 3.4%). We also observed that design GSTP1 (−158) demonstrated a statistically significant difference (p value <0.001) in detection sensitivity at 1,000 and 10,000 copies compared to designs GSTP1 (−46) and GSTP1 (−390). Therefore, all three designs show robust performance and that design GSTP1 (−158) demonstrates a small, but statistically significant, difference in analytical sensitivity.

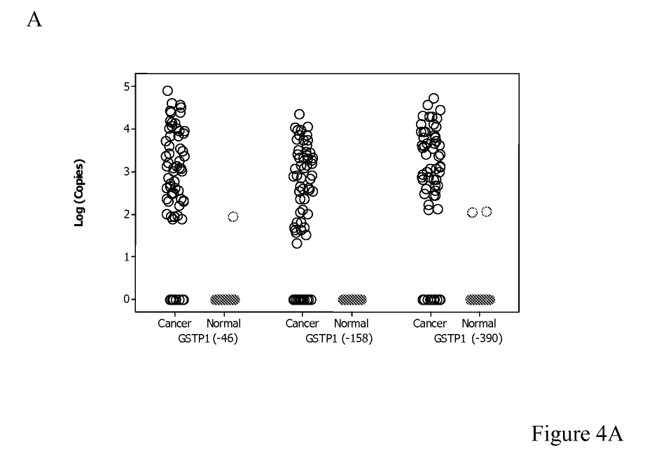

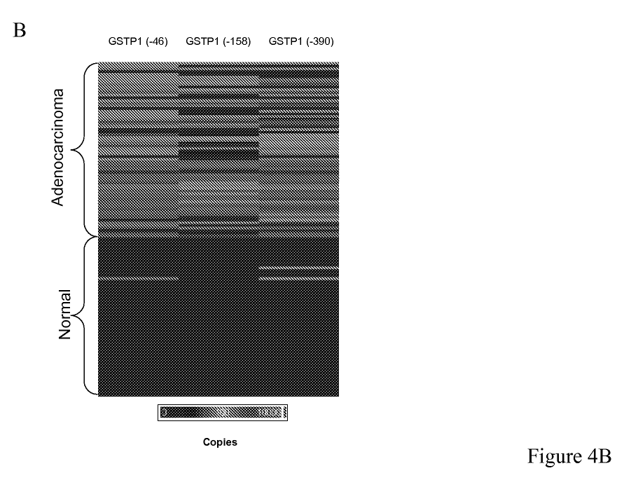

These three designs differed in their ability to detect adenocarcinomas. We tested 33 adenocarcinomas and 20 histologically negative biopsies. The data demonstrated that, although all three designs had high specificity, design GSTP1 (−390) (which was located farthest from the transcription start site) had the lowest specificity (FIG. 4A). At a specificity of 100%, the sensitivity of the three designs was 79, 76, and 78 for GSTP1 (−46), GSTP1 (−158), and GSTP1 (−390), respectively. However, using a cutoff of 10 copies, GSTP1 (−46) showed a sensitivity of 86% and a specificity of 98%, GSTP1 (−158) showed a sensitivity of 77% and a specificity of 100%, and GSTP1 (−390) showed a sensitivity of 77% and a specificity of 97%. Thus, GSTP1 (−46) demonstrated the highest clinical sensitivity with nearly equivalent specificity while GSTP1 (−158) demonstrated the highest specificity. Furthermore, the extent of methylation (as measured by the copies of methylated DNA reported) was highest for the two designs which were at the edge or closer to the edge of the CpG island (GSTP1 (−46) and GSTP1 (−390)). For example, for those adenocarcinoma samples where methylation was detected, the average and median methylation levels for GSTP1 (−46) were 7,392 copies ±13,689 and 1,339 copies and the average and median methylation levels for GSTP1 (−390) were 6,740 copies ±10,260 and 2,498 copies, compared to 2,684 copies ±4,103 and 1,163 copies for GSTP1 (−158). Lastly, the three assay designs detected overlapping subsets of adenocarcinoma samples, and the intensity of methylation differed across the designs for many samples (FIG. 4B). These data suggested that the designs could complement each other. In fact, we found that combining designs GSTP1 (−46) and GSTP1 (−390) gave a sensitivity of 91% and a specificity of 100%. We verified these findings by testing an independent set of 29 adenocarcinomas and 24 histologically negative biopsies. Once again we found that, in combination, GSTP1 (−46) and GSTP1 (−390) gave a sensitivity of 93% and a specificity of 100%. We conclude that different assay designs can demonstrate different clinical (diagnostic) sensitivities, that designs closer to the edges of a CpG island can exhibit higher levels of methylation, and that different designs can complement each other in their clinical sensitivity.

We next asked whether the different designs detected different samples due to possible correlations of a particular design (or sequence region) to clinicopathological parameters. We therefore examined the detection of adenocarcinomas over a range of Gleason scores (Table 2). We found that all three designs detected adenocarcinomas having a Gleason score of greater than 6 with a higher sensitivity than adenocarcinomas having a Gleason score below 6, although the 95% confidence intervals were overlapping for GSTP1 (−46) and GSTP1 (−390). However, GSTP1 (−158) detected adenocarcinomas having a Gleason score of greater than 6 with a higher sensitivity than adenocarcinomas having a Gleason score below 6 with a statistically significant p value (0.019). Furthermore, the difference in detection of adenocarcinomas between the three designs was more apparent for adenocarcinomas having a Gleason score below 6. Detailed epigenetic analyses of genes can therefore be used to determine cancer progression or aggressiveness.

TABLE 2

| | % Sensitivity (95% CI) | | |
|---|---|---|---|
| Gleason score | GSTP1 (−46) | GSTP1 (−158) | GSTP1 (−390) |
| <6 (N = 12) | 67 (35-90) | 42 (15-72) | 58 (28-84) |
| 6 (N = 25) | 92 (74-99) | 84 (64-95) | 80 (59-93) |
| >6 (N = 28) | 93 (76-99) | 89 (72-98) | 86 (67-96) |

Table 2 shows sensitivity of each GSTP1 design stratified according to Gleason score of the cancer.

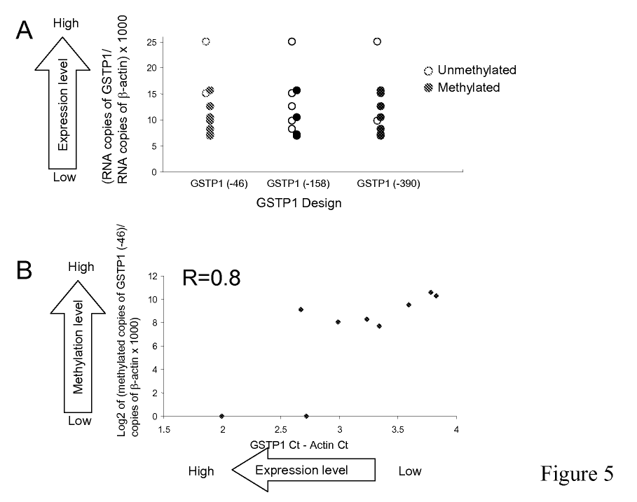

Methylation correlates with RNA expression. We used quantitative reverse transcriptase polymerase chain reaction (qRTPCR) to determine the relative expression levels of the GSTP1 transcript in nine cancer samples and compared those levels to the absence or presence of methylation of the gene in each design region. We found that, for the GSTP1 (−46) design region (located at the transcription start site), those samples which exhibited lower levels of expression were methylated in the GSTP1 gene (FIG. 5A). In contrast, the GSTP1 (−158) design did not exhibit methylation for all samples which had low levels of expression. We further examined the correlation with the GSTP1 (−46) design region by plotting the relative methylation versus relative transcript level (FIG. 5B). The data showed a correlation coefficient of 0.8. Lower correlations were observed for the other two designs. Further studies will be required to determine if the transcription start site will always show the best (inverse) correlation between expression and methylation levels.

### Discussion.

### Epigenetic Complementation Within a CpG Island.

Different assay designs for the GSTP1 gene show different sensitivities for prostate adenocarcinomas (FIG. 4). The ability of multiple assay designs for the same gene to complement each other represents a novel approach to increase sensitivity of cancer detection. This approach can be coupled with the use of multiple markers to further increase diagnostic sensitivity. First, the use of multiple designs will increase the number of individual PCR reactions performed or will require higher levels of multiplexing for single tube formats. Secondly, different designs may show different specificities for benign or normal tissues. We found that GSTP1 (−390), which was located farthest from the transcription start site, had the lowest specificity of the three designs tested (FIG. 3).

### Correlations Between Epigenetic, Transcriptomic, and Biopsy Data.

Our finding that the three designs showed inverse but different correlations with RNA expression levels (FIG. 5). Interestingly, we found that methylation at GSTP1 (−46), located at the transcription start site, exhibited the best correlation with transcription levels in both a qualitative (FIG. 5A) and quantitative (FIG. 5B) fashion.

Our finding that the frequency of methylation is higher in cancers with higher Gleason scores (Table 2) suggests that the methylation status of genes may offer a molecular description of tumor aggressiveness or other pathologic features.

In summary, complementation within a CpG island can be used to increase diagnostic sensitivity. Although all three designs demonstrated a higher sensitivity for cancers having a higher Gleason score, this bias towards higher Gleason scores did differ among the three designs. Thus, in addition to providing additional value in diagnosis, the interrogation of multiple regions of a gene could generate additional insights into prognosis.

### Materials and Methods:

### Formalin-Fixed, Paraffin-Embedded (FFPE) Radical Prostectomies and Biopsies.

A total of 66 FFPE adenocarcinoma radical prostatectomies, 36 normal tissues from radical prostatectomies, and 24 negative prostate biopsies were acquired from a variety of commercial vendors, including Asterand (Detroit, Mich.), Ardais (Lexington, Mass.) and institutional vendors in Brazil and Dr. Nagle at University of Arizona. A set of 36-paired normal and adenocarcinoma radical prostatectomies was obtained from Dr. Nagle. For each specimen, patient demographic, clinical and pathology information was collected as well. The histopathological features of each sample were reviewed to confirm diagnosis, and to estimate sample preservation and tumor content. For cancer samples, diagnoses of adenocarcinoma were unequivocally established based on histological evaluation.

DNA Isolation from FFPE Samples.

DNA isolation from paraffin tissue sections was based on the methods and reagents described in the TNES/PK protocol. Paraffin embedded tissue samples were sectioned at 5×5 μm. Sections were deparaffinized by incubation in 1 ml of xylene for 2-5 min at room temperature following a 10-20 second vortex. Tubes were then centrifuged and supernatant was removed and the deparaffinization step was repeated. After supernatant is removed 1 ml of ethanol is added and sample is vortexed for 1 minute, centrifuged and supernatant removed. This process is repeated one additional time. Residual ethanol is removed and the pellet is dried in a 55° C. oven for 5-10 minutes and resuspended in 40 μl of TNES buffer and 10 μl Proteinase K. Samples were vortexed and incubated in a thermomixer set at 500 rpm overnight at 56° C. Proteinase K within the samples was heat inactivated through incubation at 70° C. for 10 minutes. Isolated DNA either sequentially entered DNA modification or stored at −80° C. until use.

### DNA Modification.

DNA modification was based on the methods and reagents described EZ-DNA methylation kit from ZymoResearch with the following modifications. Isolated DNA was brought up to volume with the addition of 5 μl of M-dilution buffer. Samples were vortexed and incubated in a thermomixer set at 1100 rpm at 37° C. for 15 minutes followed by addition of 100 μl of CT Conversion Reagent. Tubes were then centrifuged and incubated in a thermomixer set at 1100 rpm at 70° C. for 3 hours absent of light. Bisulfite modification was suspended following a 10-minute incubation on ice. 400 μl M-binding buffer is added to each sample that is then mixed, centrifuged and the supernatant is added onto the filter column. Filter column along with collection tube are centrifuged at maximum speed for 15-30 seconds and flow through is discarded. A wash of 100 μl M-wash buffer proceed in which the solution is added to the column, centrifuged and flow through discarded. Samples were desulphonated with the addition of 200 μl of M-desulphonation buffer followed by incubation at room temperature for 15 minutes. A series of sequential washes proceed (200 μl M-wash buffer→200 μl M-wash buffer) in which each solution is added to the column, centrifuged and flow through discarded. Column is then centrifuged at maximum speed for 30 seconds, placed in a fresh 1.5 ml tube and 25 μl of elution buffer is added. Modified DNA was obtained after a 1 minute incubation at room temperature followed by a 1 minute centrifugation at maximum speed. The isolated DNA was stored in at −80° C. until use.

### Scorpion Probe and Primer Design.

Putative prostate methylation specific markers were selected as candidate marker genes for quantitative methylation specific assay. A housekeeping gene specific assay was used as an internal control to regulate the quality of the sample. Appropriate genomic DNA reference sequence accession numbers in conjunction with Visual OMP 5.0 were used to develop our quantitative methylation specific assays (prostate marker glutathione S-transferase-P1 (GSTP1) and internal control marker beta actin). Primers and Scorpion probes for these assays are listed in Table 3. Scorpion probes were labeled at the 5′ nucleotide with FAM, Texas Red and Quasar 670 as the reporter dye and at 3′ nucleotide with BHQ as the quenching dye.

TABLE 3

| | |
|---|---|
| Sequence Name | Scorpion/Primer |

| | |
|---|---|
| GSTP1_Fam_Sc_AS_1112 | FAM-CGCACGGCGAACTCCCGCCGACGTG |
| | CG BHQ-HEG-TGTAGCGGTCGTCGGGGT |
| | TG |
| GSTP1_1151_L22 | 5′ GCCCCAATACTAAATCACGACG 3′ |
| GSTP1_Sc_M_S_1207 | FAM-CCGGTCGCGAGGTTTTCGACCGG- |
| | BHQ-HEG-CCGAAAAACGAACCGCGCGTA |
| GSTP1_1179_U27 | GGGCGGGATTATTTTTATAAGGTTCGG |
| GSTP1_Sc_M_AS_888 | FAM-CGGCCCTAAAACCGCTACGAGGGCC |
| | G-BHQ-HEG-GAAGCGGGTGTGTAAGTTT |
| | CGG |
| GST_929_L26 | ACGAAATATACGCAACGAACTAACGC |
| Actin_Q670_Sc_382_L15 | Q670-CCGCGCATCACCACCCCACACGCG |
| | CGG-BHQ2-HEG-GGAGTATATAGGTTGG |
| | GGAAGTTTG |
| Actin_425_L27 | 5′ AACACACAATAACAAACACAAATTCA |
| | C 3′ |

### MSP PCR Assay.

To detect a few cancer cells, a highly sensitive and specific multiplex Methylation Specific PCR (MSP) assay is necessary. Quantitation of modified genomic DNA was carried out on the Smartcycler II (Cepheid) in 25 ul reaction. For each thermo-cycler run standard curves were amplified. Standard curves for our housekeeping markers consisted of CpGM DNA serially diluted in CpGU DNA at 100 ng, 10 ng, 1 ng and 0.1 pg. No target controls were also included in each assay run to ensure a lack of environmental contamination. MSP was carried out using PCR Buffer (46.8 mM Tris-HCl pH 8.0, 150 mM D (+) Trehalose, 5% DMSO, 0.2% Tween 20, 0.08% Proclin, 3.5 mM MgCl, 0.309 mM each of dCTP, DATP, dGTP and dTTP), Additives (2 mM Tris-Cl pH 8, 0.2 mM Albumin Bovine, 150 mM Trehalose, 0.002% Tween 20), Enzyme Mix (5 U FastStart (Roche), 46.8 mM Tris-HCl pH 8.0, 0.01% BSA, 10 mM KCl, 0.08% Proclin), Primer Mix (0.5 uM Primer, and 0.5 uM Probe). The following cycling parameters were followed: 1 cycle at 95° C. for 240 sec; and 40 cycles of 95° C. for 15 seconds, 61° C. for 30 seconds. After PCR reaction was completed baseline and threshold values were set in the Smartcycler Dx software and calculated Ct values were exported to Microsoft Excel.

For each sample, a ratio was calculated by taking the mean Ct of GSTP1 and dividing the mean Ct of β-Actin multiplied by 1000 ((Avg. Ct (GSTP1)/Avg. Ct (B-Actin)×1000)). The ratio for each GSTP1 marker set was determined for each sample. A ratio greater then zero was scored one and a ratio equal to zero was scored zero. Data was sorted according to pathological diagnosis. Parteck Pro was populated with the modified feasibility data and an intensity plot was generated.

### Quantitative RTPCR.

Quantitation of gene-specific RNA was carried out in a 96 well plate on the ABI Prism 7900HT sequence detection system (Applied Biosystems). Quantitative Real-Time PCR was performed with Taq-Man One-Step RT-PCR Master Mix Reagents (Applied Biosystems) in a 25 ul reaction containing: RT-PCR Buffer (lx Master Mix without UNG, 0.25 U/ul Multiscribe, 0.4 U/ul RNase Inhibitor), Primer and Probe Mix (0.2 uM Probe, 0.5 uM Primers). The following cycling parameters were followed: 1 cycle at 48° C. for 30 minute; 1 cycle at 95° C. for 10 minutes; and 40 cycles of 95° C. for 15 seconds, 58° C. for 30 seconds. After the PCR reaction was completed, baseline and threshold values were set in the ABI 7900HT Prism software and calculated Ct values were exported to Microsoft Excel.
References
Bastian, P et al. (2004) Molecular biomarker in prostate cancer: the role of CpG island hypermethylation Eur Urol 46:698-708
Bastian, P et al. (2007) Prognostic value of CpG island hypermethylation at PTGS2, RAR-beta, EDNRB and other gene loci in patients undergoing radical prostatectomy Eur Urol 51:665-674
Chan, Q et al. (2005) Promoter methylation and differential expression of π-class glutathione S-transferase in endometrial carcinoma J Mol Diagn 7:8-16
Coles et al. (1990) The role of glutathione and glutathione transferases in chemical carcinogenesis CRC Crit Rev Biochem Mol Biol 25:47
Das, R et al. (2006) Computational prediction of methylation status in human genomic sequences PNAS 103:10713-10716
Eckhardt, F et al. (2006) DNA methylation profiling of human chromosomes 6, 20, and 22 Nature Genetics 38:1378-1385
Endoh, M. et al. (2005) RASSF2, a potential tumor suppressor, is silenced by CpG island hypermethylation in gastric cancer Br J Cancer 93:1395-1399
Esteller, M (2005) Aberrant DNA methylation as a cancer-inducing mechanism Ann Rev Pharmacol Toxicol 45:629-656
Esteller, M et al. (2001) A gene hypermethylation profile of human cancer Cancer Res 61:3225-3229
Guo, M et al. (2006) Accumulation of promoter methylation suggests epigenetic progression in squamous cell carcinoma of the esophagus Clin Can Res 12:4515-4522
Harden, S et al. (2003) Quantitative GSTP1 Methylation and the Detection of Prostate Adenocarcinoma in Sextant Biopsies J Natl Cancer Inst 95:1634-1637
Jerónimo, C et al. (2001) Quantitation of GSTP1 Methylation in Non-neoplastic Prostatic Tissue and Organ-Confined Prostate Adenocarcinoma J Natl Cancer Inst 93:1747-1752
Jeronimo, C et al. (2004) Quantitative RARbeta2 hypermethylation: a promising prostate cancer marker Clin Cancer Res 10:4010-4014
Lee, W et al. (1994) Cytidine methylation of regulatory sequences near the pi-class glutathione S-transferase gene accompanies human prostatic carcinogenesis Proc Natl Acad Sci USA 91:11733-11737
Nakayama, M et al. (2003) Hypermethylation of the human glutathione S-transferase-π gene (GSTP1) CpG island is present in a subset of proliferative inflammatory atrophy lesions but not in normal or hyperplastic epithelium of the prostate Am J Pathol 163:923-933
Nakayama, T et al. (2001) The role of epigenetic modifications in retinoic acid receptor beta2 gene expression in human prostate cancers Lab Invest 81:1049-1057
Pao, M et al. (2001) The endothelin receptor B (EDNRB) promoter displays heterogeneous, site specific methylation patterns in normal and tumor cells Hum Molec Genet 10:903-910
Pickett et al. (1989) Glutathione S-transferases: gene structure, regulation, and biological function Annu Rev Biochem 58:743
Rushmore et al. (1993) Glutathione S-transferases, structure, regulation, and therapeutic implications J Biol Chem 268:11475
Satoh et al. (2002) DNA methylation and histone deacetylation associated with silencing DAP kinase gene expression in colorectal and gastric cancers Br J Cancer 86:1817-1823
Shaw, R et al. (2006) Promoter methylation of p16, RARβ, E-cadherin, cyclin A1 and cytoglobin in oral cancer: quantitative evaluation using pyrosequencing Br J Can 94:561-568
Stirzaker et al. (1997) Extensive DNA methylation spanning the Rb promoter in retinoblastoma tumors Cancer Res 57:2229-2237
Strunnikova, M et al. (2005) Chromatin inactivation precedes de novo DNA methylation during the progressive epigenetic silencing of the RASSF1A promoter Molec Cellular Biol 25:3923-3933
Suzuki, M et al. (2006) Methylation of apoptosis related genes in the pathogenesis and prognosis of prostate cancer Cancer Lett 242:222-230
Toyooka, S et al. (2003) Epigenetic Down-Regulation of Death-associated Protein Kinase in Lung Cancers Clin Cancer Res 9:3034-3041
Tremblay et al. (1997) A 5′ 2-kilobase-pair region of the imprinted mouse H19 gene exhibits exclusive paternal methylation throughout development Molec Cell Biol 17:4322-4329
Yan, P et al. (2003) Differential distribution of DNA methylation within the RASSF1A CpG island in breast cancer Cancer Res 63:6178-6186
Yu, J et al. (2006) DNA repair pathway profiling and microsatellite instability in colorectal cancer Clin Cancer Res 12:5104-5111

1. A method of increasing sensitivity of a DNA methylation assay comprising determining complementation within a CpG island of the methylated DNA.

2. The method of claim 1 wherein the methylation assay is for the presence of cells specific for an indication.

3. The method of claim 2 wherein the cancer, risk assessment of inherited genetic pre-disposition, identification of tissue of origin of a cancer cell such as a CTC, identifying mutations in hereditary diseases, disease status (staging), prognosis, diagnosis, monitoring, response to treatment, choice of treatment (pharmacologic), infection (viral, bacterial, mycoplasmal, fungal), chemosensitivity, drug sensitivity, metastatic potential or identifying mutations in hereditary diseases.

4. The method of claim 3 wherein the cancer is selected from breast, ovarian, lung, prostate, colon, skin, gastrointestinal or lymphatic.

5. The method of claim 4 wherein the cancer is prostate.

6. The method of claim 1 wherein the DNA methylation is associated with a gene selected from GSTP1, APC, RARβ2, HINC1.

7. The method of claim 6 wherein the DNA is in the promoter region of the gene.

8. The method of claim 7 wherein the gene is GSTP1.

9. The method of claim 8 wherein the DNA methylation is detected using sequences corresponding to those in Table 3.

10. A kit containing at least one nucleic acid molecule corresponding to the sequences in Table 3.
